(19) United States
(12) Patent Application Publication
(10) Pub. No.: US 2020/0032163 A1
(43) Pub. Date: Jan. 30, 2020

(54) Method for the Virus Inactivation of a Preparation of Monoclonal Antibodies

(71) Applicant: Laboratoire Francais du Fractionnement et des Biotechnologies, Les Ulis (FR)

(72) Inventors: Ludovic BOUCHEZ, Ales (FR); Eve DEPAUW, Saint Julien les Rosiers (FR)

(73) Assignee: Laboratoire Francais du Fractionnement et des Biotechnologies, Les Ulis (FR)

(21) Appl. No.: 16/337,004

(22) PCT Filed: Oct. 2, 2017
(86) PCT No.: PCT/EP2017/074974

§ 371 Date: Mar. 27, 2019

(30) Foreign Application Priority Data
Sep. 30, 2016 (FR) 1659449
May 22, 2017 (FR) 1754512

(51) Int. Cl. C11D 1/72; A61J 1/10; A61L 2/00; C07K 16/08

(52) U.S. Cl. CPC C11D 1/72; A61J 1/10; C07K 2317/14; C07K 16/08; A61L 2/0088

(57) ABSTRACT

The present invention relates to a method for the virus inactivation of a preparation of monoclonal antibodies, characterised in that it comprises a step of bringing the monoclonal antibody preparation into contact with a non-ionic detergent, said step being carried out while stirring in a disposable flexible pouch which can receive and contain the monoclonal antibody preparation.

### TECHNICAL FIELD

The present invention relates to a viral inactivation method of a preparation of monoclonal antibodies, as well as the virus-inactivated preparation of monoclonal antibodies.

### TECHNOLOGICAL BACKGROUND

Methods for viral inactivation were developed mainly for the production of products derived from blood. In particular, using acidic pHs, heat treatment (pasteurization), using caprylic acid, using solvent-detergent (S/D) treatment and ultraviolet (UV) irradiation are among the known methods. Due to their action, these methods make it possible to inactivate the viruses and to prevent binding or infection of the cells by these viruses. The activity of the recombinant proteins, in particular the monoclonal antibodies, is generally unaffected, because these methods target the lipids only, and not the proteins.

Viral inactivation using acidic pHs is a method commonly used during the production of monoclonal antibodies.

Pasteurization, also commonly used, consists of heating at 60° C. in a liquid medium for 10 hours. This method is considered to be effective with respect to many viruses, including HIV and hepatitis viruses.

The solvent/detergent (S/D) technique is the reference technique adopted for coagulant fractions. The simultaneous action on the preparations of coagulation factors, of an organic solvent, tri (n-butyl) phosphate (TNBP) and a detergent, sodium cholate, polysorbate 80 (Tween 80), or octoxynol (Triton X-100) destroys the viruses that have a lipid envelope. The duration of the treatment is 6 hours at temperatures from 24 to 37° C. The solvent-detergent must then be removed by adsorption chromatography, precipitation or ultrafiltration. Due to its mechanism of action, the solvent-detergent treatment acts only on the enveloped viruses.

In order to implement these methods for viral inactivation, it is known to use stainless steel containers, equipped with a double wall, in order to maintain an adequate temperature. Due to its chemical neutrality, stainless steel is not degraded by the detergents used, and makes it possible to ensure that the detergents used are not absorbed on the walls and consequently, the efficacy of the antiviral reaction.

These stainless steel containers do however have drawbacks. They require a temperature regulation system that is costly in energy and a resulting investment according to the final scale of use. In addition, use of these stainless steel containers requires cleaning and sterilizing of the equipment between each batch of product, which generally results in extending the production time and adding to the production line.

As a result, a need arises to have a viral inactivation method that is simple to use, flexible, economical in energy and inexpensive, making it possible to reduce the risks of cross-contamination and also capable of inactivating the enveloped viruses contained in a preparation of monoclonal antibodies.

### DETAILED DESCRIPTION

The applicant has shown, surprisingly, that viral inactivation of a preparation of monoclonal antibodies can be carried out in single-use flexible bags. Despite the chemically non-neutral character of the walls of these bags, the applicant has demonstrated, surprisingly, that the nonionic detergents used are not absorbed on the walls of the bag and allow effective inactivation of the viruses present in the preparation of monoclonal antibodies. In addition, the applicant has shown, surprisingly, that the detergents used do not degrade the walls of the flexible bags and therefore do not lead to leaching of any contaminants. This step of viral inactivation of a preparation of monoclonal antibodies in single-use flexible bags makes it possible in particular to save time (absence of cleaning) and energy (no need for temperature regulation) compared with the use of the stainless steel containers of the state of the art.

The applicant of the present invention therefore proposes a novel viral inactivation method comprising a step of bringing a preparation of monoclonal antibodies into contact with a nonionic detergent, the contacting being carried out under stirring in a single-use flexible bag.

A subject of the invention is thus a viral inactivation method of a preparation of monoclonal antibodies, characterized in that it comprises a step of bringing the preparation of monoclonal antibodies into contact with a nonionic detergent, the contacting being carried out under stirring in a single-use flexible bag capable of receiving and containing the preparation of monoclonal antibodies.

Within the meaning of the present invention, by “viral inactivation method” is meant any method making it possible to inactivate, remove or eliminate certain types of viruses that are potentially present in the preparation of monoclonal antibodies. Advantageously, the viruses inactivated by the method according to the invention are in particular the enveloped viruses, such as viruses related to the xenotropic murine leukemia virus (X-MuLV), bovine viral diarrhoea virus (BVDV) complex, pseudorabies virus (PRV). Advantageously, the viral inactivation method according to the invention does not use a solvent. In other words, the viral inactivation method according to the invention making it possible to inactivate, remove or eliminate certain types of viruses that are potentially present in the preparation of monoclonal antibodies, without using or adding solvent. The applicant has shown, surprisingly, that using a nonionic detergent alone made it possible to inactivate, remove or eliminate certain types of viruses that are potentially present in the preparation of monoclonal antibodies, in a particularly effective manner. Advantageously, no solvent, such as tri-n-butyl phosphate or TnB is used or added in the viral inactivation method according to the invention.

Within the meaning of the present invention, the term “antibody” refers to a molecule of immunoglobulin or a fragment of a molecule of immunoglobulin having the ability to bind specifically to a particular antigen. The term antibody also includes different types of antibodies, for example human antibodies, recombinant antibodies, monoclonal antibodies, humanized antibodies, chimeric antibodies, fragments of antibodies F(ab′)₂, Fab, scFv, Fv or also a mixture of these antibodies. In a particular embodiment of the invention, the antibody is obtained by any technique well known to a person skilled in the art. In particular, the antibody can be obtained from human serum or from different sources, in particular from cells, plants or non-human animals, or also by genetic engineering.

According to another particular aspect of the invention, the antibodies are recombinant antibodies. The term “recombinant antibody” as used here includes the antibodies that are prepared, expressed, created or isolated by recombinant means, such as antibodies isolated from an animal that is transgenic for the immunoglobulin genes of another species, antibodies expressed using a recombinant expression vector transfected into a host cell, isolated antibodies from a library of combinatorial recombinant antibodies, or antibodies prepared, expressed, created or isolated by any other means that involves splicing immunoglobulin gene sequences with other DNA sequences.

According to yet another particular aspect, the antibodies can be chimeric or humanized. According to the present invention, the term “chimeric antibody” refers to an antibody that combines parts of non-human antibodies (for example mouse, rat, rabbit), with parts of human antibodies. According to the present invention, the term “humanized antibody” refers to an antibody that retains only the CDR regions binding to the antigen of the parent antibody, in combination with human framework regions (see Waldmann, 1991, Science 252:1657). Such humanized or chimeric antibodies containing the specific binding sites of the murine antibody are expected to have reduced immunogenicity when they are administered in vivo for diagnostic purposes, for prophylactic or therapeutic applications according to the invention.

According to another aspect, the antibodies are human antibodies. The term “human antibodies” as used here, includes antibodies having variable and constant regions derived from human immunoglobulin germline sequences. The human antibodies according to the invention can also include amino acid residues non-coded by human immunoglobulin germline sequences (for example, mutations introduced at random, or by site-specific in vitro mutagenesis, or by in vivo somatic mutation. Human antibodies are generally generated using transgenic mice bearing parts of the human immune system rather than that of mice. Fully human monoclonal antibodies can also be prepared by immunizing transgenic mice with large parts of the heavy and light chains of human immunoglobulins, in particular according to the techniques described in American patents U.S. Pat. Nos. 5,591,669, 5,598,369, 5,545,806, 5,545,807, 6,150,584. These animals have been genetically modified so that there is a functional deletion in the production of endogenous antibodies (for example murine). The animals are also modified to contain all or a portion of the locus of the human germline immunoglobulin gene so that the immunization of these animals results in the production of fully human antibodies directed against the antigen of interest.

Depending on the immunization of these mice (for example XenoMouse (Abgenix), HuMAb mice (Medarex/GenPharm)), the monoclonal antibodies are prepared according to the conventional hybridoma techniques. These monoclonal antibodies have human immunoglobulin amino acid sequences and thus do not cause human anti-mouse antibody (HAMA) responses when they are administered to humans. As with any antibody according to the present invention, the human antibodies can be monoclonal antibodies.

According to another particular aspect, the antibody is a full-length antibody. According to yet another particular aspect, this full-length antibody comprises a light chain and a heavy chain.

According to a particular embodiment, the antibody is produced in a transgenic non-human mammal, in particular in a female goat, a sheep, bison, camel, cow, pig, rabbit, horse, rat, mouse or llama.

Advantageously, the antibody according to the invention is a therapeutic recombinant monoclonal antibody. Advantageously, the therapeutic recombinant monoclonal antibody has a therapeutic activity and may be capable either of neutralizing a soluble antigen (for example: toxin, cytokine, virus), or of specifically targeting a membrane receptor with a view to blocking the binding between the ligand and this receptor, or of having a cytotoxic effect.

Within the meaning of the present invention, by “capable of receiving and containing the preparation of monoclonal antibodies” is meant a bag capable of receiving and containing the preparation of monoclonal antibodies according to the invention and which complies with the criteria specified by the European Pharmacopoeia. Advantageously, a bag is considered to be capable of receiving and containing the preparation of monoclonal antibodies if it has a satisfactory mechanical strength after filling thereof with the preparation of monoclonal antibodies and is chemically compatible with the preparation of monoclonal antibodies.

Within the meaning of the present invention, by “flexible bag” is meant a closed envelope formed from a material that can be deformed when it is subjected to pressure or a stress, for example during filling or stirring of the bag. Such a material can for example remain flexible at a temperature comprised between 5° C. and 60° C., advantageously at a temperature comprised between 15° C. and 25° C. Advantageously, the flexibility of the bag makes it possible to avoid the risks of opening if dropped.

In an advantageous embodiment, the flexible bag according to the invention is single-use. By “single-use” is meant a bag that is disposable after use and serves for one single use only. Within the meaning of the present invention, by “use” is meant any possible use of the bag and advantageously the intravenous administration of the antibodies to patients in need thereof. The single-use flexible bag advantageously makes it possible to dispense with the cleaning processes that are often lengthy and troublesome, unlike the use of stainless steel containers. Thus, the single-use flexible bag makes it possible to gain time and flexibility during the process of production of the preparation of monoclonal antibodies and is less expensive in terms of investment.

In an advantageous embodiment, the flexible bag (1) according to the invention is constituted by a multilayer film of polymers (2) and a stirring means (3), By “multilayer” is meant within the meaning of the present invention, a succession of layers. Advantageously, the film according to the invention comprises at least two layers, advantageously at least three layers and even more advantageously at least four layers. In an advantageous embodiment, the multilayer film of polymers (2) comprises four layers of different composition. In an advantageous embodiment, the multilayer film of polymers (2) is constituted by four different layers made from plastic material. In an advantageous embodiment, the multilayer film of polymers (2) is constituted by the following polymers: polyethylene terephthalate (PET) or polyamide (PA) or ethylene vinyl alcohol copolymer (EVOH) or polyethylene (PE), or a mixture of at least 2 of said polymers. In an advantageous embodiment, the multilayer film of polymers (2) comprises: an outer layer constituted by polyethylene terephthalate (PET), a first intermediate layer constituted by polyamide (PA), a second intermediate layer constituted by ethylene vinyl alcohol copolymer (EVOH), and an inner layer constituted by polyethylene (PE), said inner layer being directly in contact with the preparation of monoclonal antibodies. Advantageously, the outer layer constituted by polyethylene terephthalate acts as a strong transparent protective barrier, allowing light to pass through. Polyethylene provides strength to the film and contributes to reducing the gaseous exchanges through the film. Advantageously, the first intermediate layer constituted by polyamide makes it possible to increase the hardness and to reinforce the structure of the bag. This first intermediate layer also makes it possible to reduce the gaseous exchanges through the film. Advantageously, the second intermediate layer constituted by ethylene vinyl alcohol copolymer acts as a main barrier to the gases, thus minimizing the transmission of gases such as oxygen and carbon dioxide through the film. Advantageously, the inner layer constituted by polyethylene is directly in contact with the preparation of monoclonal antibodies. The polyethylene used in this inner layer complies with the usual Pharmacopoeia specifications and constitutes a contact layer that is clean, inert and highly chemically resistant. According to an advantageous embodiment, the four layers of the multi-layer membrane are assembled together by an adhesive layer. In a particularly advantageous embodiment, the flexible bag according to the invention can be that described in patent EP 1012227.

In another particularly advantageous embodiment, the flexible bag according to the invention can be a bag such as that described in patent EP 0941158. In particular, the bag according to the invention can be constituted by a multilayer structure comprising: a core layer of an ethylene vinyl alcohol copolymer having an ethylene content from 25 to 45 mole percent; an inner layer constituted by an ethylene homopolymer or a copolymer of ethylene and an alpha-olefin, suitable for contact with a solution and placed on a first side of the core layer; an outer layer placed on a second side of the core layer opposite to the inner layer, the outer layer being constituted by a polyimide; and two tie-layers, one bonded to the first side and one to the second side of the core layer and placed between the inner layer and the core layer and between the outer layer and the core layer; in which the core layer, the inner layer, the outer layer and the tie-layers are coextruded by moulding on top of one another, and in which the inner layer is thicker than the outer layer.

In another particularly advantageous embodiment, the flexible bag according to the invention can be a bag such as that described in patent EP 1426178. In particular, the bag according to the invention can be constituted by a multilayer structure comprising: a core layer of an ethylene vinyl alcohol copolymer having an ethylene content from 25 to 45 mole percent; an inner layer composed of a copolymer of ethylene and a-olefin, and placed on a first side of the core layer; an outer layer placed on a second side of the core layer opposite to the inner layer, the outer layer being composed of a polymer selected from the group constituted by polyamides, polyesters and polyolefins; and two tie-layers, one bonded to the first side and one to the second side of the core layer and placed between the inner layer and the core layer and between the outer layer and the core layer; in which the core layer, the inner layer, the outer layer and the two tie-layers are coextruded to one another, in which the structure has a water-soluble fraction soluble in water at low molecular weight of less than 1.0 parts per thousand (ppt).

In an advantageous embodiment according to the invention, the flexible bag (1) can be transparent, translucent or opaque. Advantageously, the flexible bag (1) can be transparent.

In an advantageous embodiment according to the invention, the flexible bag (1) according to the invention also comprises one or more inlet openings (4), allowing the preparation of monoclonal antibodies to be introduced during the filling of said bag. Advantageously, the flexible bag (1) according to the invention can comprise at least one, advantageously at least two, advantageously at least three, advantageously at least four, advantageously at least five, advantageously at least six, advantageously at least seven inlet openings (4). In an advantageous embodiment according to the invention, the flexible bag (1) according to the invention also comprises one or more outlet openings (7). Advantageously, the flexible bag (1) according to the invention can comprise at least one, advantageously at least two, advantageously at least three, advantageously at least four, advantageously at least five, advantageously at least six, advantageously at least seven outlet openings (7).

In an advantageous embodiment according to the invention, the thickness of the multilayer film of polymers (2) is comprised between 100 and 300 μm, advantageously between 150 and 250 μm, advantageously between 200 and 250 μm. Even more advantageously, the thickness of the multilayer film of polymers (2) is comprised between 200 and 225 μm.

In an advantageous embodiment according to the invention, the inner and outer layers have a thickness comprises between 6 μm and 20 μm.

In an advantageous embodiment, the flexible bag is equipped with a stirring means (3). Advantageously, the stirring means (3) is a stirring device.

Advantageously, the stirring device is a spiral stirrer. This spiral stirrer can be coupled with a device for driving in rotation by levitation, said device for driving in rotation by levitation being able to be outside the flexible bag and itself being driven in rotation by a motor rotor.

In particular, the stirring device can be mounted rotatably on a support (5) arranged inside said flexible bag, on the inner layer (6) of the multilayer film of polymers constituting the flexible bag, said layer being directly in contact with the preparation of monoclonal antibodies. Said support can be fastened on said face, for example removably, for example by means of a suction cup.

The device for driving in rotation can be a part of the Flexel® system marketed by Sartorius. In an advantageous embodiment, a flexible bag according to the invention can be the Flexel® Bags bag, marketed by Sartorius.

Advantageously, the duration of stirring is greater than or equal to 20 minutes, advantageously greater than or equal to 30 minutes, advantageously greater than or equal to 60 minutes, and more advantageously greater than or equal to 120 minutes. Advantageously, each flexible bag can be provided with said stirring system for the mixture of nonionic detergent/preparation of monoclonal antibodies. Advantageously, the means for stirring the nonionic detergent/preparation of monoclonal antibodies mixture are means arranged in order to drive a rotary stirring of said bag.

In an advantageous embodiment, the holding capacity of the single-use flexible bag is comprised between 5 millilitres and 2000 litres, advantageously a maximum holding capacity comprised between 50 millilitres and 1500 litres, advantageously comprised between 75 millilitres and 1250 litres, advantageously comprised between 100 millilitres and 1000 litres, advantageously comprised between 200 millilitres and 750 litres, advantageously comprised between 300 millilitres and 500 litres, advantageously comprised between 400 millilitres and 250 litres, advantageously comprised between 500 millilitres and 125 litres, advantageously comprised between 750 millilitres and 100 litres, advantageously comprised between 1 litre and 75 litres, advantageously comprised between 2 litres and 50 litres, advantageously comprised between 5 litres and 25 litres. Advantageously, the bag has a maximum holding capacity of 75 millilitres. More advantageously, the bag has a maximum holding capacity of 500 millilitres. Even more advantageously, the bag has a maximum holding capacity of 50 litres. In another embodiment of the invention, the bag has a maximum holding capacity of 2000 litres.

In an advantageous embodiment according to the invention, the method comprises a step of bringing the preparation of monoclonal antibodies into contact with a nonionic detergent in the flexible bag. In a particularly advantageous embodiment, the duration of the contact is comprised between 20 minutes and 72 hours, advantageously comprised between 20 minutes and 24 hours, advantageously comprised between 20 minutes and 6 hours, advantageously between 30 minutes and 5 hours, advantageously between 30 minutes and 4 hours, advantageously between 30 minutes and 3 hours. Advantageously, the duration of contact is comprised between 30 minutes and 2 hours. Advantageously, the duration of contact is comprised between 60 minutes and 90 minutes. In an advantageous embodiment according to the invention, the step of bringing the preparation of monoclonal antibodies into contact with a nonionic detergent in the flexible bag is carried out in the absence of solvent. In a particularly advantageous embodiment according to the invention, the step of bringing the preparation of monoclonal antibodies into contact with a nonionic detergent in the flexible bag is carried out in the absence of tri-n-butyl phosphate or TnBP. In other words, the step of bringing the preparation of monoclonal antibodies into contact with a nonionic detergent in the flexible bag does not involve tri-n-butyl phosphate or TnBP, Advantageously, the step of bringing the preparation of monoclonal antibodies into contact with a nonionic detergent in the flexible bag is carried out without adding solvent. In a particularly advantageous embodiment according to the invention, the step of bringing the preparation of monoclonal antibodies into contact with a nonionic detergent in the flexible bag is carried out without adding tri-n-butyl phosphate or TnBP. In other words, the step of bringing the preparation of monoclonal antibodies into contact with a nonionic detergent in the flexible bag does not involve tri-n-butyl phosphate or TnBP. The step of bringing the preparation of monoclonal antibodies into contact is only carried out with a nonionic detergent in the flexible bag, without adding solvent.

In an advantageous embodiment according to the invention, the step of bringing the preparation of monoclonal antibodies into contact with a nonionic detergent is carried out at a temperature comprised between 2° C. and 35° C., advantageously between 13° C. et 35° C., advantageously between 20° C. and 35° C., advantageously between 20° C. and 30° C. Even more advantageously, the step of bringing the preparation of monoclonal antibodies into contact with a nonionic detergent is carried out at an ambient temperature comprised between 15° C. and 25° C. In an advantageous embodiment according to the invention, the step of bringing the preparation of monoclonal antibodies into contact with a nonionic detergent in the flexible bag is carried out in the absence of solvent. In a particularly advantageous embodiment according to the invention, the step of bringing the preparation of monoclonal antibodies into contact with a nonionic detergent in the flexible bag is carried out in the absence of tri-n-butyl phosphate or TnBP. In other words, the step of bringing the preparation of monoclonal antibodies into contact with a nonionic detergent in the flexible bag does not involve tri-n-butyl phosphate or TnBP. Advantageously, the step of bringing the preparation of monoclonal antibodies into contact with a nonionic detergent in the flexible bag is carried out without adding solvent. In a particularly advantageous embodiment according to the invention, the step of bringing the preparation of monoclonal antibodies into contact with a nonionic detergent in the flexible bag is carried out without adding tri-n-butyl phosphate or TnBP. In other words, the step of bringing the preparation of monoclonal antibodies into contact with a nonionic detergent in the flexible bag does not involve tri-n-butyl phosphate or TnBP. The step of bringing the preparation of monoclonal antibodies into contact is only carried out with a nonionic detergent in the flexible bag, without adding solvent.

In an advantageous embodiment according to the invention, the contact surface area between the multilayer film of polymers and the preparation of monoclonal antibodies/nonionic detergent mixture is comprised between 0.2 cm²/mL et 3.5 cm²/mL, advantageously between 0.2 cm²/mL et 1.2 cm²/mL. Even more advantageously, the contact surface area between the multilayer film of polymers and the preparation of monoclonal antibodies/nonionic detergent mixture is 0.2 cm²/mL.

In an advantageous embodiment according to the invention, the detergent used is a nonionic detergent selected from polyethylene glycol sorbitan monolaurate (also called polysorbate 20, marketed in particular under the name of Tween 20®), polyethylene glycol monooleate (also called polysorbate 80 or marketed in particular under the name Tween 80e), polyethylene glycol and octophenyl ether (polyethylene glycol tert-octylphenyl ether) marketed under the name Triton X-100® or 4-(1,1,3,3-tetramethylbutyl)phenyl-polyethylene glycol or also t-octylphenoxypolyethoxyethanol, nonyl phenoxypolyethoxyethanol (marketed under the name NP-40®), 3-[(3-cholamidopropyl)dimethylammonio]propanesulphonic acid (marketed under the name CHAPS® ) and n-dodecyl-β-D-maltoside (also called DDM). Advantageously, the nonionic detergent used must be chemically compatible with the bag according to the invention, and in particular, the nonionic detergent must not alter the structure of the bag, i.e. must not alter the transparency of the bag, must not colour it, must not render the laminate film friable, nor crack it, must not cause damage to the surfaces of the bag, such as in particular decomposing or delaminating it, nor cause bubbles to appear in the film.

In a particularly advantageous embodiment, the nonionic detergent is polyethylene glycol tert-octylphenyl ether, i.e. Triton X-100®. Polyethylene glycol tert-octylphenyl ether (Triton X-100®) has the advantage of being chemically compatible with the multilayer film of polymers, which constitutes the flexible bag. In fact, the inventors have shown that polyethylene glycol tert-octylphenyl ether fully met the criterion of chemical compatibility, to the extent that it is not absorbed by the multilayer film of polymers and in that the latter does not alter the structure of the multilayer film of polymers, i.e. that polyethylene glycol tert-octylphenyl ether has no negative impact on the thickness or the transparency of the film, nor on the colouration of the film. In addition, no degradation of the film was observed, nor any alteration of the resistive strength of the film or of the openings present on the bag.

In an advantageous embodiment according to the invention, the final concentration of the nonionic detergent is greater than or equal to 0,7%, advantageously greater than or equal to 0.9%, advantageously greater than or equal to 1%, advantageously less than or equal to 1.2%, the percentage being expressed in weight-for-weight (w/w). Even more advantageously, the final concentration of the nonionic detergent is 1%, the percentage being expressed in weight-for-weight (w/w).

In a particularly advantageous embodiment, the viral inactivation method according to the invention does not use a solvent. In a particularly advantageous embodiment according to the invention, the viral inactivation method according to the invention does not use or involve tri-n-butyl phosphate or TnBP.

In an advantageous embodiment of the invention, the viral inactivation method comprises:
a) a step of bringing a preparation of monoclonal antibodies into contact with a nonionic detergent, the contacting being carried out under stirring in a single-use flexible bag capable of receiving and containing the preparation of monoclonal antibodies,
b) a step of removing the nonionic detergent,
c) a step of recovering the virus-inactivated preparation of monoclonal antibodies.

In a particularly advantageous embodiment, the viral inactivation method comprises:
a) a step of bringing a preparation of monoclonal antibodies into contact with a nonionic detergent, the contacting being carried out under stirring and in the absence of solvent, in a single-use flexible bag capable of receiving and containing the preparation of monoclonal antibodies,
b) a step of removing the nonionic detergent,
c) a step of recovering the virus-inactivated preparation of monoclonal antibodies.

In an advantageous embodiment of the invention, step a) is carried out in the absence of solvent. In other words, step a) is carried out without solvent, but only with a nonionic detergent. In a particularly advantageous embodiment according to the invention, step a) is carried out without tri-n-butyl phosphate or TnBP.

In an advantageous embodiment of the invention, step b) of removing the nonionic detergent can be implemented by any method well known to a person skilled in the art. Advantageously, step b) of removing the nonionic detergent can be carried out using a chromatography support, advantageously using a chromatography support comprising an ion-exchange resin, advantageously using a chromatography support comprising an anion or cation exchange resin. Step b) of the viral inactivation method makes it possible to directly remove the nonionic detergent in a fraction that is not retained.

In an advantageous embodiment of the invention, step c) of recovering the virus-inactivated preparation of monoclonal antibodies can be implemented by eluting the preparation of monoclonal antibodies from the chromatographic support.

In a particularly advantageous embodiment of the invention, the viral inactivation method comprises:
a) a step of bringing the preparation of monoclonal antibodies into contact with a nonionic detergent, the contacting being carried out under stirring in a single-use flexible bag capable of receiving and containing the preparation of monoclonal antibodies,
b) a step of removing the nonionic detergent using a chromatography support comprising an ion exchange resin,
c) a step of recovering the virus-inactivated preparation of monoclonal antibodies by eluting the preparation of monoclonal antibodies from the chromatography support.

In a particularly advantageous embodiment of the invention, the viral inactivation method comprises:
a step of bringing the preparation of monoclonal antibodies into contact with a nonionic detergent, the contacting being carried out under stirring and in the absence of solvent, in a single-use flexible bag capable of receiving and containing the preparation of monoclonal antibodies,
b) a step of removing the nonionic detergent using a chromatography support comprising an ion exchange resin.
c) a step of recovering the virus-inactivated preparation of monoclonal antibodies by eluting the preparation of monoclonal antibodies from the chromatography support.

Advantageously, step a) of the viral inactivation method is carried out in the absence of solvent. In other words, step a) is carried out without solvent, but only with a nonionic detergent. In a particularly advantageous embodiment according to the invention, step a) is carried out without tri-n-butyl phosphate or TnBP.

In an even more advantageous embodiment of the invention, the viral inactivation method comprises:
a) a step of bringing a preparation of monoclonal antibodies into contact with polyethylene glycol tern-octylphenyl ether, the contacting being carried out under stirring in a single-use flexible bag capable of receiving and containing the preparation of monoclonal antibodies,
b) a step of removing the nonionic detergent using a chromatography support comprising an ion exchange resin,
c) a step of recovering the virus-inactivated preparation of monoclonal antibodies by eluting the preparation of monoclonal antibodies from the chromatography support.

In an advantageous embodiment of the invention, the viral inactivation method comprises:

a step of bringing a preparation of monoclonal antibodies into contact with polyethylene glycol tert-octylphenyl ether, the contacting being carried out under stirring and in the absence of solvent, in a single-use flexible bag capable of receiving and containing the preparation of monoclonal antibodies,

b) a step of removing the nonionic detergent using a chromatography support comprising an ion exchange resin,

c) a step of recovering the virus-inactivated preparation of monoclonal antibodies by eluting the preparation of monoclonal antibodies from the chromatography support.

Advantageously, step a) of the viral inactivation method is carried out in the absence of solvent. In other words, step a) is carried out without solvent, but only with polyethylene glycol tert-octylphenyl ether. In a particularly advantageous embodiment according to the invention, step a) is carried out without tri-n-butyl phosphate or TnBP.

In an advantageous embodiment of the invention, the inactivation method makes it possible to obtain a viral reduction factor greater than 3 log, preferably greater than 4 log, preferably greater than 5 log, with respect to the viral load initially present in the preparation of monoclonal antibodies. In a particularly advantageous embodiment of the invention, bringing the preparation of recombinant monoclonal antibodies into contact with polyethylene glycol tert-octylphenyl ether (Triton X-100®) in the single-use flexible bag as described above, makes it possible to reduce the viral load initially contained in the preparation of monoclonal antibodies by a factor of at least 3 log for the enveloped viruses.

In an even more advantageous embodiment, bringing the preparation of recombinant monoclonal antibodies into contact with polyethylene glycol tert-octylphenyl ether (Triton X-100®) in the single-use flexible bag as described above makes it possible to reduce the viral load initially contained in the preparation of monoclonal antibodies by a factor of at least 4 log for the X-MuLV virus, at least 5 log for the BVDV virus and at least 5 log for the pseudorabies virus (PRV),

Another aspect of the invention relates to an virus-inactivated preparation of monoclonal antibodies obtained according to the viral inactivation method described above. In an advantageous embodiment, the virus-inactivated preparation of monoclonal antibodies is obtained by bringing the preparation of monoclonal antibodies into contact with a nonionic detergent, advantageously polyethylene glycol tert-octylphenyl ether (Triton))X-100®) the contacting being carried out under stirring and without adding solvent, in a single-use flexible bag capable of receiving and containing the preparation of monoclonal antibodies. Advantageously, the viral load present in said virus-inactivated preparation of monoclonal antibodies is reduced by a factor of at least 4 log for the enveloped viruses, with respect to the viral load initially contained in the preparation of monoclonal antibodies.

### FIGURES

FIG. 1: Diagrammatic representation of an embodiment of a flexible bag according to the invention.

FIG. 2: Determining the concentration of adsorbed Triton X-100® according to Example 2, expressed in mg/L as a function of the incubation time expressed in minutes, for the flexible bag according to the invention and a glass flask.

FIG. 3: Determining the concentration of adsorbed Triton X-100® according to Example 4, expressed in mg/L as a function of the incubation time expressed in minutes, for the flexible bag according to the invention and a glass flask.

FIG. 4: Determining the reduction factor of the viral load of the X-MULV and BVDV viruses, expressed as a log reduction, for the flexible bag (Bag) according to the invention and a stainless steel container (Vessel). Detergent alone is used in the flexible bag and a conventional mixture of solvent-detergent is used in the stainless steel container.

FIG. 5: Determining the concentration of adsorbed polysorbate 80 according to Example 6, expressed in mg/L as a function of the incubation time expressed in hours, for the flexible bag according to the invention and a glass flask.

FIG. 6: Determining the concentration of adsorbed TnBP according to Example 6, expressed in mg/L as a function of the incubation time expressed in hours, for the flexible bag according to the invention and a glass flask.

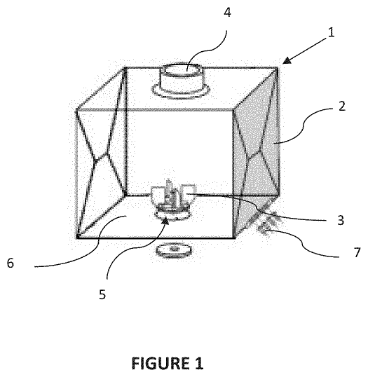

### EXAMPLES

### Example 1

### Viral Inactivation Method

This study was carried out using samples obtained from the processes for the production of two different monoclonal antibodies mAb A and mAb B. The mAb A and mAb B samples were taken from eluates resulting from the step of affinity chromatography having a resin based on protein A. The efficacy of the treatment with the detergent Triton X-100® for inactivating the enveloped viruses was assessed according to the ICH Guidelines and the European Agency for the Evaluation of Medicinal Products (EMEA) Guidelines ((ICH Q5A (2009); EMEA (1996)). The target concentration of Triton X-100® is 1%, and the specified limits are 0.9 and 1.2%. The proteins concentration of the two mAb A and mAb B samples is less than 10 g/L. The viral inactivation method was tested for the following viruses: xenotropic murine leukemia virus (XMuLV), bovine viral diarrhoea virus complex (BVDV), pseudorabies virus (PRV), The tests were carried out using a bag of 75 ml capacity. The experimental conditions implemented are presented in Table 1 below.

TABLE 1
Experimental conditions

| | Operating | Minimum | Minimum |
|---|---|---|---|
| | conditions | operating | operating |
| | at industrial | conditions for | conditions for |
| Parameters | scale | mAbA | mAbB |
| Final concentration of | 1 | 0.7 | 0.7 |
| Triton X-100 ® (% | | | |
| (w/w)) | | | |
| Incubation time | ≥1 | 1 | 1 |
| (hours) | | | |
| Temperature (° C.) | 20 ± 5 | 14 ± 1° C. | 14 ± 1 |
| Stirring | Stirred | Stirred | Stirred |

A viral load of 5% (v/v) was added to the mAb A and mAb B samples. A sample was taken in order to determine the initial viral load contained in each of the samples. The loaded mAb A and mAb B samples were mixed with Triton X-100® having a final concentration of 0.7% (w/w) and placed under stirring in the Flexel® system.

The mAb A and mAb B samples treated with the detergent Triton X-100® were taken at the following different times: 0-1 minute, 5 minutes, 30 minutes and 1 hour, then diluted immediately before titration. All the tests were carried out using the 50% Tissue Culture Infective Dose method (TCID₅₀) for measuring infectiousness.

The negative control is a loaded sample to which no detergent had been added. No statistically significant change was observed in the measurement of the infectiousness of the control sample.

Results

The results are presented in Table 2.

TABLE 2
Reduction factor of the viral load obtained in eluates from chromatography on protein
A for two monoclonal antibodies mAb A and mAb B treated with Triton X-100 ®.

| | | | | Viral load in the fractions | | | | |
|---|---|---|---|---|---|---|---|---|
| | | | | collected after treatment | | | | |
| | | | Initial viral load | (log10 TCID₅₀/mL) | | | | |
| | Temperature | | before treatment | T = | T = | T = | T = | Reduction |
| | (° C.) | Virus | (log10 TCID₅₀/mL) | 0-1 min | 5 min | 30 min | 1 hr | factor (log) |
| mAb A | 17 | X-MuLV | 6.63 | 3.83 | NT | 3.83 | 1.45 | ≥5.18 ± 0.36 |
| | 14 ± 1 | X-MuLV | 6.19 | 3.83 | 3.83 | 3.83 | 1.45 | ≥4.74 ± 0.32 |
| mAb B | 14 ± 1 | X-MuLV | 5.76 | 3.83 | 3.83 | 3.83 | 1.92 | 3.84 ± 0.47 |
| | 14 ± 1 | X-MuLV | 5.67 | 3.83 | 3.83 | 3.83 | 1.55 | ≥4.12 ± 0.22 |
| | 14 ± 1 | BVDV | 6.63 | 3.83 | 3.83 | 3.83 | 1.45 | ≥5.18 ± 0.20 |
| | 14 ± 1 | BVDV | 6.81 | 3.83 | 3.83 | 3.83 | 1.45 | ≥5.36 ± 0.29 |
| | 14 ± 1 | PRV | 6.72 | 3.83 | 3.83 | 3.83 | 1.45 | ≥5.27 ± 0.24 |
| | 14 ± 1 | PRV | 6.46 | 3.83 | 3.83 | 3.83 | 1.45 | ≥5.01 ± 0.34 |

A reduction was observed in the viral load of the X-MuLV virus of a minimum of 4.74 log for the mAb A sample treated with the detergent Triton X-100® for 1 hour.

A reduction was observed in the viral load of the X-MuLV virus of a minimum of 3.84±0.47 log for the mAb B sample treated with the detergent Triton X-100® for 1 hour,

A reduction was observed in the viral load of the BVDV virus greater than 5.18±0.20 log for the mAb B sample treated with the detergent Triton X-100® for 1 hour.

A reduction was observed in the viral load of the PRV virus greater than 5.01±0.34 log for the mAb B sample treated with the detergent Triton X-100® for 1 hour.

These results confirm that the viral inactivation method is effective to the extent that the reduction in the viral load observed is greater than 4 log.

### Example 2

### Study of the Rate of Adsorption of the Multilayer Film of Polymers of the Flexible Bag during Treatment with the Detergent Triton X-100®

A single-use flexible bag according to the invention, having a maximum holding capacity of 75 mL was filled with 63 mL of Triton X-100® solution at a concentration of 1% (w/w). The bag containing the detergent was placed on an orbital stirring device in order to create a movement of the liquid without however generating a vortex or foam. The bag was then incubated for 120 minutes at ambient temperature (20±5° C.). Samples were periodically taken after incubating for 0 min, 30 min, 60 min and 120 min and the concentration of Triton X-100® was measured by high-performance liquid-phase chromatography (HPLC).

In parallel, a negative control was carried out, repeating the experiment in a 0.5 litre glass flask filled with 316 mL of a 1% Triton X-100® solution. The solution was stirred using a magnetic mixer and a magnetic stirring bar. Samples were taken after 0 and 120 minutes.

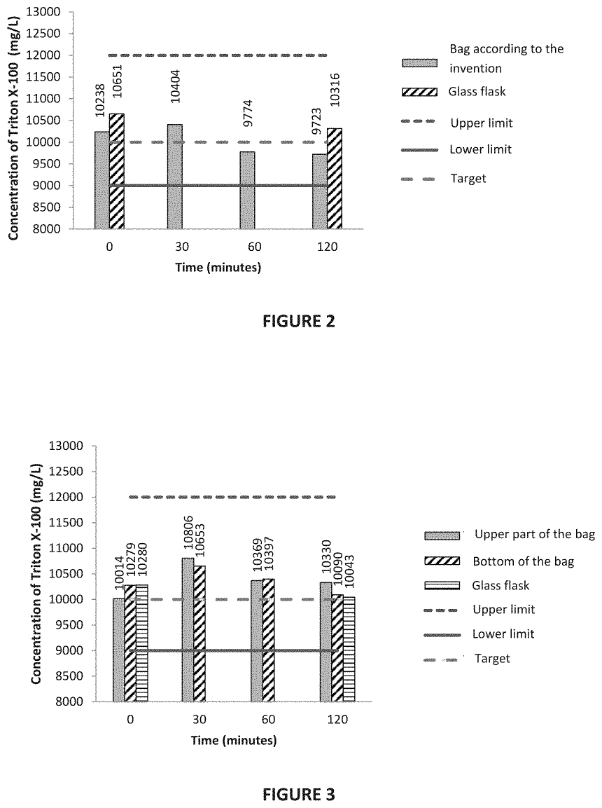

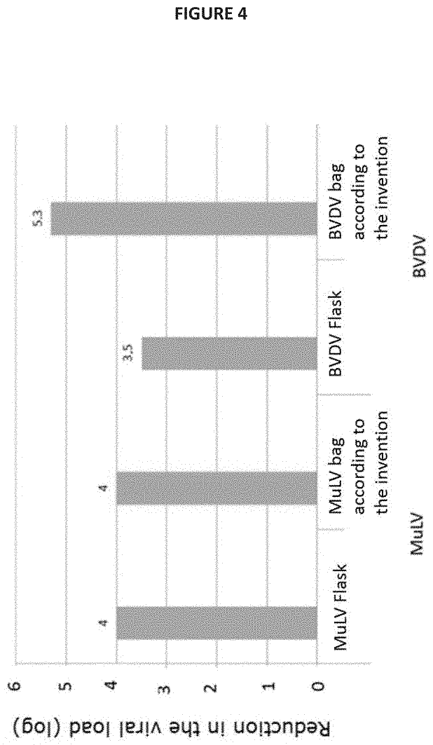

The results are presented in FIG. 2.

As presented in FIG. 2, the concentration of Triton X-100® reduced by 5% in the bag and 3.1% in the glass flask after incubating for more than 120 minutes. In both cases, the final concentration of Triton X-100® is comprised within the acceptable concentration range of 9 g/L to 12 g/L. This experiment shows that there is no significant adsorption of the Triton X-100® by the bag. This confirms that the bag of the invention is suitable for implementing the viral inactivation step using Triton X-100®.

### Example 3

### Study of the Chemical Compatibility and the Leachable Substances for a Bag having a Capacity of 500 mL

Study of Chemical Compatibility

The mechanical strength of the flexible bag according to the invention was assessed using a Triton X-100® solution at a concentration of 20%. The bag containing the detergent was then incubated under stirring for 72 hours, at a temperature of 5° C.±3° C. The bag used has a holding capacity of 500 mL, the contact surface area between the multilayer film of polymers and the detergent is 1.2 cm²/mL. Before the bag was brought into contact with the detergent, the bag was irradiated by means of gamma radiation at 25-45 kGy. A negative control was also prepared by replacing the Triton X-100® solution with purified water.

In order to demonstrate the chemical compatibility of the bag with the detergent, a set of tests was carried out after incubation with the detergent. A visual inspection makes it possible to assess the impact of the solution on the plastic material, criteria such as transparency, friability of the film, the presence of cracking, the presence of bubbles, surface damage such as decomposition and delamination, were also assessed. An analysis of weight loss was carried out in order to measure any potential reduction in the barrier properties of the bag. The mechanical properties were tested by a traction test on the multilayer film of polymers, on the sealing elements as well as on the connections, and compared with the specifications. The stress test conditions were applied by means of a drop test in order to assess the mechanical strength of the bag after being brought into contact with a 20% Triton X-100® solution.

The interaction between the components and the detergent was also assessed by verifying that the dimensions of the main components were still compliant with the required specifications after being brought into contact with the test solution. Finally, the spectral properties of the film were verified and compared with the negative control.

Study of Leachable Substances

The study of leachable substances was carried out by bringing the flexible bag according to the invention into contact with a Triton X-100® solution at a concentration of 1%. The bag containing the detergent was then incubated under stirring for 24 hours, at a temperature of 30° C.±5° C. The bag used has a holding capacity of 500 mL.

A negative control was also prepared by replacing the flexible bag with a glass flask and a polytetrafluouroethylene (PTFE) flask of the same capacity,

Analysis of the impurities (inorganic elements) was carried out by inductively coupled plasma mass spectrometry (ICP-MS) in order to assess the migration of the inorganic elements into the plastic materials (35 elements were assessed, according to the ICH Q3D, EMEA and USP Guidelines, section <232>). The volatile and semi-volatile leachable substances were analysed using gas chromatography coupled with mass spectrometry (GC-MS). The organic compounds reactive to UV were analysed using high-performance liquid-phase chromatography coupled with an ultraviolet spectrum detector (HPLC-UV), independently of their molecular weight.

Results Obtained:

Study of Chemical Compatibility

The results of the study of chemical compatibility of the flexible bag when the latter is brought into contact under stirring with a Triton X-100® solution at a concentration of 20%, for 72 hours, at a temperature of 5° C.±3° C. are presented in Table 3 below.

TABLE 3
Results obtained for the chemical compatibility of the flexible
bag having a capacity of 500 mL brought into contact with a
Triton X-100 ® solution at a concentration of 20%.

| Test | | |
|---|---|---|
| category | Description of test | Result of test |
| N/A | Analysis of the bag weight loss | Satisfactory |
| Category 1 | Visual inspection of the bag: transparency/ | Satisfactory |
| | opacity | |
| | Visual inspection of the bag: colour change | Satisfactory |
| Category 2 | Thickness of the film | Satisfactory |
| | Dimension of the spiral | Satisfactory |
| Category 3 | Resistance to traction of the film | Satisfactory |
| Category 4 | Visual inspection of the bag: formation of | Satisfactory |
| | bubbles | |
| | Visual inspection of the bag: delamination | Satisfactory |
| Category 5 | Verification of leakage at the accessories | Satisfactory |
| | Verification of leakage at the seams of the | Satisfactory |
| | bag | |
| | Verification of leakage at the film | Satisfactory |
| | Verification of leakage at the inlet opening | Satisfactory |
| | Visual inspection of the bag: decomposition | Satisfactory |
| | Visual inspection of the bag: friability | Satisfactory |
| | Visual inspection of the bag: crater/hole | Satisfactory |

The results of the study of chemical compatibility show that the flexible bag according to the invention has an excellent chemical compatibility when it is exposed to a 20% Triton X-100® solution for 72 hours at a temperature of 5° C.±3° C.

Study of leachable Substances

The results of the study of leachable substances are presented in Table 4 below.

TABLE 4
Results obtained for the study of leachable substances of the
flexible bag having a capacity of 500 mL brought into contact
with a Triton X-100 ® solution at a concentration of 1%.

| Analyses | Method | Results |
|---|---|---|
| Impurities | ICP-MS\|OES | Potassium and zinc were detected at a |
| (inorganic | | quantifiable level equivalent to that observed |
| elements) | | for the negative controls |
| Organic | RP-HPLC-UV | No leachable substances detected |
| leachable | | |
| substances | | |
| Organic | GC-MS | Two leachable substances of the hydrocarbon |
| leachable | | type were detected at a concentration of |
| substances | | approximately 1 ppm each |

### Example 4

### Study of the Distribution of the Concentration of Triton X-100® in a bag having a Capacity of 50 L.

This study was carried out in order to verify that the concentration of Triton X-100® is homogenous inside the flexible bag once the latter is stirred by the magnetic stirring device. The adsorption as well as the efficacy of the stirring were verified. The study was carried out with purified water. A flexible bag according to the invention having a capacity of 50 L was filled with 50 L of purified water. The detergent Triton X-100® at a concentration of 1% was added. The bag containing water and the Triton X-100® was subjected to stirring at a speed of 40 rpm. Samples were taken after incubating for 0, 30, 60 and 120 minutes, at a point that is 30 mm below the surface of the liquid and at the level of the bottom of the bag. In this way, the concentration of Triton X-100® was assessed both at the top and the bottom of the bag.

For the purposes of the present experiment, the first appearance of a homogenous solution following stirring is considered to be time T=0.

A control was carried out in parallel in a 5 L borosilicate glass bottle with 2105 g of 1% Triton X-100® in purified water. The solution was stirred using a plate-like mixing bar and a magnetic stirring device, at a speed of 100 rpm. Samples were taken after incubating for 0 and 120 minutes.

Sampling was carried out using 30 mL sterile glass flasks that were immediately stored at 2-8° C. The concentration of Triton X-100® was measured by reverse-phase high-performance liquid chromatography (RP-HPLC).

Results Obtained:

The results obtained are presented in FIG. 3.

The difference in concentration of Triton X-100® between the upper part and the bottom of the bag is 2.7%, which is less than the value of 10% for the difference in concentration of Triton X-100® between the upper part and the bottom of the bag. Thus, the ability of the magnetic stirring device to distribute the concentration of Triton X-100® homogenously within the bag was demonstrated.

The concentration of Triton X-100® was considered as stable over time in the bag according to the invention, to the extent that the greatest difference observed between the lower and higher concentrations measured is 7.90%, which is comprised within the specified limits ranging from 0 to 10%.

### Example 5

### Method for the Viral Inactivation of the MLV Virus by Solvent/Detergent (SID) Treatment

The solvent/detergent (SID) solution was prepared from 126 g/L polysorbate 80 and 38.5 g/L. of TnBP. The SID solution was diluted to 70%, by adding 30 g of water for injectable preparations in a glass recipient under continuous stirring. This solution was stored at ambient temperature. This solution was used for epilation experiments (condition 1 and condition 2).

The viral inactivation method by SID treatment was tested on the murine leukemia virus (MLV) at two different temperatures:
test 1: 14±1° C.
test 2: 18±1° C.

The experimental conditions implemented are presented in Table 5.

TABLE 5
Experimental protocol for the preparation of the S/D solution

| Parameters | Test 1: | Test 2: |
|---|---|---|
| Contents | Single-use bag | Single-use bag |
| | according | according |
| | to the invention | to the invention |
| Temperature (° C.) | 14 ± 1° C. | 18 ± 1° C. |
| Incubation time (hours) | 6 | 6 |
| Incubation conditions | under stirring | under stirring |
| Concentration of | 0.7 | 0.7 |
| Polysorbate 80 (expressed | | |
| as % (w/v) | | |
| Concentration of TnBP | 0.21 | 0.21 |
| (expressed as % (v/v) | | |

A viral load (murine leukemia virus (MLV)) of 5% (v/v) was added to the solution of monoclonal antibodies. A sample was taken in order to determine the initial viral load contained in the sample. The sample containing the solution of loaded monoclonal antibodies was mixed with the S/D solution diluted to 70% prepared beforehand and placed at two different temperatures: 14±1° C. for test 1 or 18±1° C. for test 2.

A sample of the solution of monoclonal antibodies treated with the S/D solution was taken at the following different times: 30 minutes, 1 hour, 2 hours, 3 hours. 4 hours, 5 hours and 6 hours, then diluted immediately before titration for each of the two temperatures tested. All the tests were carried out using the 50% Tissue Culture Infective Dose method (TCID₅₀).

The negative control is a loaded sample to which no solvent/detergent has been added. No statistically significant change was observed in the infectiousness of the control sample.

Results

The results are presented in Tables 6 and 7.

TABLE 6
Results obtained by S/D treatment at 14 ± 1° C.

| | | Initial viral | Viral load in the | | |
|---|---|---|---|---|---|
| | | load before | fractions collected | | Clearance |
| | Viral load | treatment | after treatment | Reduction | factor ± |
| | (log10 | (log10 | (log10 | factor ± | CL |
| Sample | TCID₅₀/mL) | TCID₅₀/mL) | TCID₅₀/mL) | CL (log 10) | (log 10) |
| T = 30 min | 7.08 ± 0.31 | 7.24 ± 0.38 | 3.91 ± 0.18 | 3.33 ± 0.42 | 3.17 ± 0.36 |
| T = 1 h | 7.08 ± 0.31 | 7.24 ± 0.38 | 3.57 ± 0.25 | 3.67 ± 0.45 | 3.51 ± 0.40 |
| T = 2 h | 7.08 ± 0.31 | 7.24 ± 0.38 | 3.15 ± 0.21 | 4.09 ± 0.43 | 3.93 ± 0.37 |
| T = 3 h | 7.08 ± 0.31 | 7.24 ± 0.38 | 1.13 ± 0.49 | 6.11 ± 0.62 | 5.95 ± 0.58 |
| T = 4 h | 7.08 ± 0.31 | 7.24 ± 0.38 | 1.13 | >6.11 ± 0.38 | >5.95 ± 0.31 |
| T = 5 h | 7.08 ± 0.31 | 7.24 ± 0.38 | 1.13 | >6.11 ± 0.38 | >5.95 ± 0.31 |
| T = 6 h | 7.08 ± 0.31 | 7.24 ± 0.38 | 1.13 | >6.11 ± 0.38 | >5.95 ± 0.31 |

TABLE 7
Results obtained by S/D treatment at 18 ± 1° C.

| | | Initial viral | | | |
|---|---|---|---|---|---|
| | | load before | Viral load in the | | |
| | Viral load | treatment | fractions collected | Reduction | Clearance |
| | (log10 | (log10 | after treatment | factor ± CL | factor ± CL |
| Sample | TCID₅₀/mL) | TCID₅₀/mL) | (log10 TCID₅₀/mL) | (log 10) | (log 10) |
| T = 30 min | 7.08 ± 0.31 | 6.81 ± 0.29 | 3.10 ± 0.65 | 3.71 ± 0.71 | 3.98 ± 0.72 |
| T = 1 h | 7.08 ± 0.31 | 6.81 ± 0.29 | 1.56 ± 0.30 | 5.25 ± 0.42 | 5.52 ± 0.43 |
| T = 2 h | 7.08 ± 0.31 | 6.81 ± 0.29 | 1.13 | >5.68 ± 0.29 | >5.95 ± 0.31 |
| T = 3 h | 7.08 ± 0.31 | 6.81 ± 0.29 | 1.13 | >5.68 ± 0.29 | >5.95 ± 0.31 |
| T = 4 h | 7.08 ± 0.31 | 6.81 ± 0.29 | 1.13 | >5.68 ± 0.29 | >5.95 ± 0.31 |
| T = 5 h | 7.08 ± 0.31 | 6.81 ± 0.29 | 1.13 | >5.68 ± 0.29 | >5.95 ± 0.31 |
| T = 6 h | 7.08 ± 0.31 | 6.81 ± 0.29 | 1.13 | >5.68 ± 0.29 | >5.95 ± 0.31 |

A reduction was observed in the viral load of the MLV virus of a minimum of 6.11 log for the solution of monoclonal antibodies treated with the SID solution (0.7% (w/v)) polysorbate 80+0.21% (v/v) TnBP) after 4 hours of treatment at 14±1° C.

A reduction was observed in the viral load of the MLV virus of a minimum of 5.68 log for the solution of monoclonal antibodies treated with the SID solution (0.7% (w/v)) polysorbate 80+0.21% (v/v) TnBP) after 2 hours of treatment at 18±1° C.

### Example 6

### Study of the Rate of Adsorption of the Multilayer Film of Polymers of the Flexible Bag during Treatment with the Solvent/Detergent Solution (TnBP/Polysorbate 80)

Two single-use flexible bags according to the invention, having a maximum holding capacity of 75 mL were filled respectively with 65 mL of TnBP solution (solvent) at a concentration of 1% (v/v) and with 65 mL of polysorbate 80 solution (detergent) at a concentration of 0.3% (w/v). The bag containing the detergent was placed on an orbital stirring device in order to create a movement of the liquid but without thereby generating a vortex or foam.

The bags were then incubated for 120 minutes at ambient temperature (25±3° C.). Samples were periodically taken after incubating for 0 min, 30 min, 1 hour and 2 hours and the concentration of solvent (TnBP) and detergent (polysorbate 80) was measured by high-performance liquid-phase chromatography (HPLC).

A negative control was carried out in parallel, repeating the experiment in two glass flasks filled respectively with, in the first flask, a TnBP solution (solvent) at a concentration of 1% (v/v) and, in the second, a polysorbate 80 solution (detergent) at a concentration of 0,3% (w/v). The solution was stirred using a magnetic mixer and a magnetic stirring bar. Samples were taken after 30 minutes.

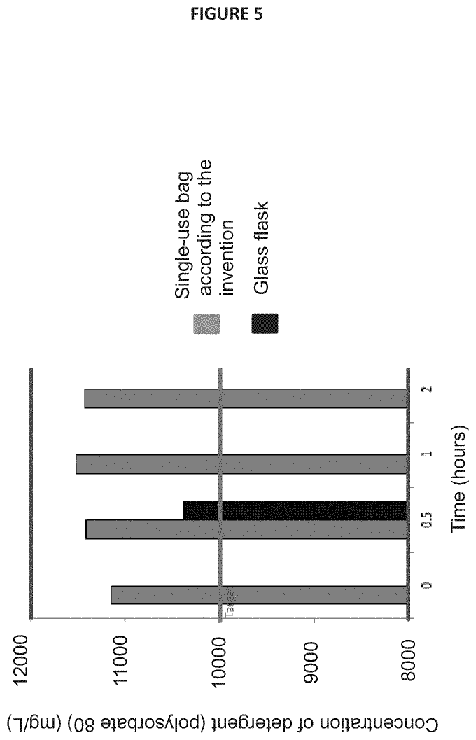

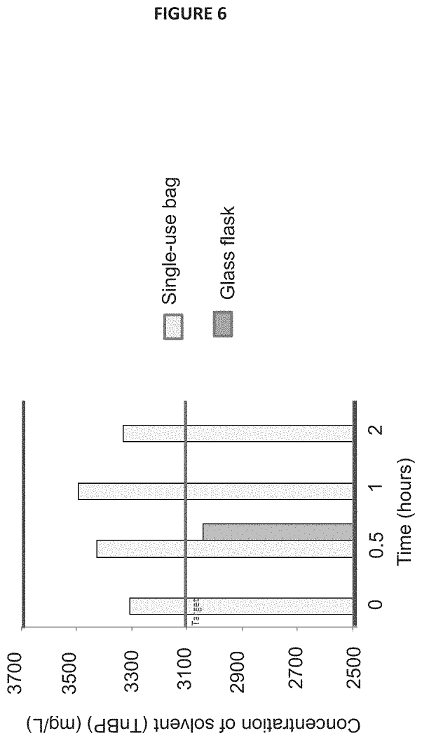

The results are presented in FIGS. 5 and 6 and in Table 8.

TABLE 8
Comparison of the adsorption data of polysorbate 80
and of TnBP as a function of the content.

| | | Polysorbate 80 | | TnBP (mg/L) | |
|---|---|---|---|---|---|
| | | | Adsorption | | Adsorption |
| | | Concentration | with respect | Concentration | with respect |
| Contents | Time | (mg/L) | to T0 (%) | (mg/L) | to T0 (%) |
| Glass | 30 min | 10374 | NA | 3042 | NA |
| Bag | 0 min | 11147 | NA | 3308 | NA |
| according | | | | | |
| to the | | | | | |
| invention | | | | | |
| Bag | 30 min | 11408 | −2.3 | 3425 | −3.5 |
| according | | | | | |
| to the | | | | | |
| invention | | | | | |
| Bag | 1 hour | 11519 | −3.3 | 3493 | 5.6 |
| according | | | | | |
| to the | | | | | |
| invention | | | | | |
| Bag | 2 hours | 11432 | −2.6 | 3333 | 0.8 |
| according | | | | | |
| to the | | | | | |
| invention | | | | | |

As presented in FIG. 5, the concentration of polysorbate 80 is stable, whether in the bag or in the glass flask, after 2 hours of incubation.

This experiment shows that there is no significant adsorption of the polysorbate 80 by the bag. This confirms that the bag according to the invention is suitable for implementing the viral inactivation step using polysorbate 80.

As presented in FIG. 6, the concentration of TnBP was reduced by 0.8% in the bag, after incubating for 2 hours.

This experiment shows that there is no significant adsorption of the TnBP by the bag. This confirms that the bag of the invention is suitable for implementing the viral inactivation step using TnBP.

1. A method for viral inactivation method of a preparation of monoclonal antibodies, comprising contacting the preparation of monoclonal antibodies with a nonionic detergent, wherein the contacting is carried out under stirring in a single-use flexible bag capable of receiving and containing the preparation of monoclonal antibodies.

2. The viral inactivation method according to claim 1, wherein the flexible bag comprises a multilayer film of polymers and a stirrer.

3. The viral inactivation method according to claim 2, wherein the stirrer is a spiral stirrer.

4. The viral inactivation method according to claim 1, wherein the nonionic detergent is selected from polyethylene glycol sorbitan monolaurate, polyethylene glycol sorbitan monooleate, polyethylene glycol tert-octylphenyl ether, nonyl phenoxypolyethoxylethanol, 3-[(3-cholamidopropyl)dimethylammonio]propanesulphonic acid, and n-dodecyl-β-D-maltoside.

5. The viral inactivation method according to claim 4, wherein the nonionic detergent is polyethylene glycol tert-octylphenyl ether.

6. The viral inactivation method according to claim 1, wherein the step of bringing the preparation of monoclonal antibodies is contacted with the nonionic detergent in the absence of solvent.

7. A virus-inactivated preparation of monoclonal antibodies obtained according to the method of claim 1.
